(19) United States
(12) Patent Application Publication
(10) Pub. No.: US 2019/0030491 A1
(43) Pub. Date: Jan. 31, 2019

(54) METHODS FOR PREPARING CARBON MOLECULAR SIEVE HOLLOW FIBER MEMBRANES FOR GAS SEPARATION

(71) Applicant: Georgia Tech Research Corporation, Atlanta, GA (US)

(72) Inventors: Chen Zhang, Atlanta, GA (US); William John Koros, Atlanta, GA (US)

(21) Appl. No.: 16/039,936

(22) Filed: Jul. 19, 2018

Related U.S. Application Data
(60) Provisional application No. 62/536,678, filed on Jul. 25, 2017.

(51) Int. Cl. B01D 67/00; B01D 53/22; B01D 71/02; B01D 69/08; C07C 7/144; C10L 3/10; C01B 13/02; C01B 21/04; C01B 32/50

(52) U.S. Cl. CPC B01D 67/0067; B01D 53/228; B01D 71/021; B01D 69/088; C07C 7/144; C10L 3/102; C01B 2210/0046; C01B 21/0438; C01B 32/50; B01D 2053/224; B01D 2323/40; C10L 2290/548; C01B 13/0251

(57) ABSTRACT

In embodiments of the present disclosure, a CMS hollow fiber membranes may be prepared to have an ultrathin (e.g. 2 microns or less) separation layer. A precursor hollow fiber may be prepared as dual layer fibers having a thin sheath layer and a core layer. During pyrolysis, the sheath layer is transformed into an ultrathin separation layer. Porosity of the core layer substrate is well-maintained during pyrolysis, thereby enabling high permeance of the CMS hollow fiber membrane. Additionally, in some embodiments, the sheath layer of the precursor hollow fibers may be hybridized prior to pyrolysis. By hybridizing the sheath layer prior to pyrolysis, a CMS hollow fiber may having an improved separation factor, including for example increased carbon dioxide/methane selectivity, may be provided.

This application claims priority to U.S. Provisional Patent Application No. 62/536,678, filed Jul. 25, 2017, the entirety of which is incorporated by reference herein.

### SUMMARY OF THE INVENTION

Embodiments of the present disclosure relate to methods to form carbon molecular sieve (CMS) hollow fiber membranes with attractive selectivity and substantially increased permeance properties, by pyrolysis of enhanced precursor hollow fibers with ultrathin sheath layers.

In some embodiments, the CMS hollow fiber membranes may be prepared to have ultrathin (e.g. 2 microns or less) separation layers. For instance, the precursor hollow fibers may be prepared as dual layer fibers having a sheath layer and a core layer. Such dual layer fibers may be spun by co-extruding two polymer solutions (dopes). The precursor hollow fibers may be prepared with optimized compositions and spinning parameters. For example, in some embodiments, one may add polyvinylpyrrolidone (PVP) in the core spin dope. Through control over the dope composition and the spinning process, precursor hollow fibers having ultrathin sheath layers and substantially increased support substrate porosity may be formed. During pyrolysis, the ultrathin sheath layer is transformed into the ultrathin separation layer of the CMS hollow fiber membrane. Porosity of the core layer substrate is well-maintained during pyrolysis, thereby enabling high permeance of the CMS hollow fiber membrane.

In some embodiments, the sheath layer of the precursor hollow fibers may be hybridized prior to pyrolysis. By hybridizing the sheath layer of the precursor hollow fibers with engineered polyamide, polyimide, or polyamide-imide prior to pyrolysis (i.e. pre-pyro hybridization), the CMS hollow fibers showed improved separation factors, including for example increased carbon dioxide/methane selectivity.

CMS hollow fiber membranes prepared from hybridized precursor fibers and having an ultrathin separation layer showed substantially increased carbon dioxide permeances and attractive carbon dioxide/methane separation factors at 35 degrees Celsius.

### BRIEF DESCRIPTION OF THE DRAWINGS

A clear conception of the advantages and features of one or more embodiments will become more readily apparent by reference to the exemplary, and therefore non-limiting, embodiments illustrated in the drawings:

FIG. 1 is an illustration showing a conventional dry-jet/wet-quench spinning process for preparing polymer hollow fibers.

FIG. 2 is an SEM image of the outer portion and surface of a conventional precursor polymer hollow fiber.

FIG. 3 is an SEM image of the outer portion and surface of a CMS hollow fiber pyrolyzed from the precursor shown in FIG. 2, after treatment of the precursor with a modifying agent that has been shown to restrict substructure collapse, showing a separation layer having a thickness of about 5-6 microns.

FIG. 4 is an SEM image of the outer portion and surface of an embodiment of a dual-layer precursor polymer hollow fiber prepared according to the present disclosure, showing a distinct core layer and sheath layer (−1 μm).

FIG. 5 is an SEM image of the outer portion and surface of a CMS hollow fiber pyrolyzed from the dual-layer precursor shown in FIG. 4, after treatment of the precursor with a modifying agent that has been shown to restrict substructure collapse, showing a separation layer having a thickness of about 1 μm.

FIG. 6 is an SEM image of the outer portion and surface of an embodiment of a dual-layer precursor polymer hollow fiber prepared according to the present disclosure, showing a distinct core layer and sheath layer (˜400 nm).

FIG. 7 is an SEM image of the outer portion and surface of a CMS hollow fiber pyrolyzed from the dual-layer precursor shown in FIG. 6, after treatment of the precursor with a modifying agent that has been shown to restrict substructure collapse, showing a separation layer having a thickness of about 400 nm.

FIG. 8 is a schematic illustration showing hybridization of the sheath layer of a dual-layer precursor fiber or the skin layer of an asymmetric single-layer precursor fiber, as well as formation of a defect-free CMS hollow fiber membrane.

FIG. 9 is a schematic illustration showing formation of a hybridized skin layer on top of a precursor fiber with porous surface, as well as formation of a defect-free CMS hollow fiber membrane.

FIG. 10 is a schematic illustration showing the ring-opening reaction of the polyimide precursor fiber by the amine functional group of the post-spinning polyamide (or polyimide) coating. The reaction can occur in the sheath layer of a dual-layer precursor fiber, the skin layer of an asymmetric single-layer precursor fiber, or inside the pores of a precursor fiber with porous surface.

### DETAILED DESCRIPTION OF THE INVENTION

Membranes are widely used for the separation of gases and liquids, including for example, separating acid gases, such as CO₂ and H₂S from natural gas. Gas transport through such membranes is commonly modeled by the sorption-diffusion mechanism. Specifically, gas molecules sorb into the membrane at the upstream, and finally desorb from the membrane at the downstream. Two intrinsic properties are commonly used to evaluate the performance of a membrane material; “permeability” and “selectivity.” Permeability is hereby defined as a measure of the intrinsic productivity of a membrane material; more specifically, it is the partial pressure and thickness normalized flux, typically measured in Barrer. Permeance, which is sometimes used in place of permeability, measures the pressure-normalized flux of a given compound. Selectivity, meanwhile, is a measure of the ability of one gas to permeate through the membrane versus a different gas; for example, the permeability of CO₂ versus CH₄, measured as a unit-less ratio.

Currently, polymeric membranes are well studied and widely available for gaseous separations due to easy processability and low cost. CMS membranes, however, have been shown to have attractive separation performance properties exceeding that of polymeric membranes. CMS membranes are typically produced through thermal pyrolysis of polymer precursors. Many polymers have been used to produce CMS membranes in fiber and dense film form. Polyimides have a high glass transition temperature, are easy to process, and have one of the highest separation performance properties among other polymeric membranes, even prior to pyrolysis.

Because carbon molecular sieve (CMS) membranes can be prepared to selectively permeate a first gas component from a gas mixture, they may be used for a wide range of gas separation applications. For instance, in various embodiments, CMS membranes may be configured for the separation of particular gases, including but not limited to CO₂ and CH₄, H₂S and CH₄, CO₂/H₂S and CH₄, CO₂ and N₂, O₂ and N₂, N₂ and CH₄, He and CH₄, H₂ and CH₄, H₂ and C₂H₄, ethylene and ethane, propylene and propane, and ethylene/propylene and ethane/propane, each of which may be performed within a gas stream comprising additional components. One of the many gas separation applications in which CMS membranes may be particularly suitable is in the separation of acid gas components—CO₂, H₂S, or a combination thereof—from a hydrocarbon-containing gas stream such as natural gas. The CMS membranes may be prepared so as to selectively sorb these acid gases, producing a permeate stream having an increased concentration of acid gases and a retentate stream having a reduced concentration of acid gases.

Substrate collapse is a major challenge for carbon molecular sieve (CMS) hollow fiber membranes. Asymmetric hollow fiber's porous substrate is densified during high-temperature pyrolysis. Thus, the hollow fiber membrane completely or partially loses its asymmetry and has increased separation layer thickness with unattractive membrane permeance. For example, the skin layer thickness of a conventional asymmetric Matrimid® (Tg˜305° C.) precursor hollow fiber is usually below 1 μm. After pyrolysis at or above 550° C., however, the porous substrate is totally collapsed and the fiber wall entirely densified. The resulting CMS hollow fiber has an effective separation layer thickness equal to the fiber wall thickness (usually 30-50 μm) and the membrane permeance is unattractive.

Treating the precursor hollow fibers with modifying agents, as is described in U.S. Pat. No. 9,211,504 B2 and U.S. Pat. Application No. 14/501,884 (published as US 2015/0094445 A1), the entireties of which are incorporated herein by reference, has been shown to restrict substrate collapse and reduce the separation layer thickness to 4-6 μm, which is still much thicker than the skin layer of the precursor fiber. To maximize membrane permeance, it is of great interest to further reduce CMS fiber skin thickness below 4 μm, and ideally to reduce it to a level comparable with precursor skin thickness (e.g., about 1 μm).

Embodiments of the present disclosure are directed to methods to minimize CMS hollow fiber substrate collapse by manipulating the structure of the precursor hollow fiber prior to pyrolysis. In particular, by spinning dual-layer precursor hollow fiber membranes with controllable sheath layer thickness/morphology and increased support substrate porosity, ultrathin CMS hollow fiber membranes were formed with substantially reduced separation layer thickness (e.g. less than 2 μm). By hybridizing the precursor hollow fiber, and in particular the sheath layer of a dual-layer precursor fiber, prior to pyrolysis, the ultrathin CMS hollow fiber membranes showed substantially increased permeances and attractive separation factors.

### Asymmetric Hollow Fiber Membranes

Carbon molecular sieve (CMS) membranes have shown great potential for the separation of gases, such as for the removal of carbon dioxide (CO₂) and other acid gases from natural gas streams. Asymmetric CMS hollow fiber membranes are preferred for large scale, high pressure applications.

Asymmetric hollow fiber membranes have the potential to provide the high fluxes required for productive separation due to the reduction of the separating layer to a thin integral skin on the outer surface of the membrane. The asymmetric hollow morphology, i.e. a thin integral skin supported by a porous base layer or substructure, provides the fibers with strength and flexibility, making them able to withstand large transmembrane driving force pressure differences. Additionally, asymmetric hollow fiber membranes provide a high surface area to volume ratio.

Asymmetric CMS hollow fiber membranes comprise a thin and dense skin layer supported by a porous substructure. Asymmetric polymeric hollow fibers, or precursor fibers, are conventionally formed via a dry-jet/wet-quench spinning process, also known as a dry/wet phase separation process or a dry/wet spinning process. The precursor fibers are then pyrolyzed at a temperature above the glass transition temperature of the polymer to prepare asymmetric CMS hollow fiber membranes.

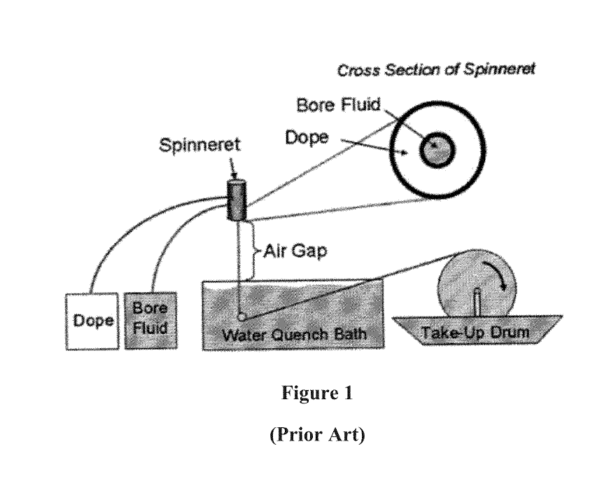

The polymer solution used for spinning of an asymmetric hollow fiber is referred to as dope. During spinning of a conventional precursor polymer fiber, the dope surrounds an interior fluid, which is known as the bore fluid. The dope and bore fluid are coextruded through a spinneret into an air gap during the “dry-jet” step. The spun fiber is then immersed into an aqueous quench bath in the “wet-quench” step, which causes a wet phase separation process to occur. After the phase separation occurs, the fibers are collected by a take-up drum and subjected to a solvent exchange process. An example of this process is shown in FIG. 1.

A conventional solvent exchange process involves two or more steps, with each step using a different solvent. The first step or series of steps involves contacting the precursor fiber with one or more solvents that are effective to remove the water in the membrane. This generally involves the use of one or more water-miscible alcohols that are sufficiently inert to the polymer. The aliphatic alcohols with 1-3 carbon atoms, i.e. methanol, ethanol, propanol, isopropanol, and combinations of the above, are particularly effective as a first solvent. The second step or series of steps involves contacting the fiber with one or more solvents that are effective to replace the first solvent with one or more volatile organic compounds having a low surface tension. Among the organic compounds that are useful as a second solvent are the C₅ or greater linear or branched-chain aliphatic alkanes.

The solvent exchange process typically involves soaking the precursor fibers in a first solvent for a first effective time, which can range up to a number of days, and then soaking the precursor fibers in a second solvent for a second effective time, which can also range up to a number of days. Where the precursor fibers are produced continuously, such as in a commercial capacity, a long precursor fiber may be continuously pulled through a series of contacting vessels, where it is contacted with each of the solvents. The solvent exchange process is generally performed at room temperature.

The precursor fibers are then dried by heating to temperature above the boiling point of the final solvent used in the solvent exchange process and subjected to pyrolysis in order to form asymmetric CMS hollow fiber membranes.

The choice of polymer precursor, the formation and treatment of the precursor fiber prior to pyrolysis, and the conditions of the pyrolysis all influence the performance properties of an asymmetric CMS hollow fiber membrane.

### Pyrolysis Conditions

Pyrolysis is advantageously conducted under an inert atmosphere. In some embodiments, the pyrolysis temperature may be between about 500° and about 1000° C., alternatively the pyrolysis temperature may be between about 500° and about 900° C.; alternatively, the pyrolysis temperature may be between about 500° and about 800° C.; alternatively, the pyrolysis temperature may be between about 500° and about 700° C.; alternatively, the pyrolysis temperature may be between about 500° and 650° C.; alternatively, the pyrolysis temperature may be between about 500° and 600° C.; alternatively, the pyrolysis temperature may be between about 500° and 550° C.; alternatively, the pyrolysis temperature may be between about 550° and about 700° C.; alternatively, the pyrolysis temperature may be between about 550° and about 650° C. alternatively the pyrolysis temperature may be between about 600° and about 700° C.; alternatively the pyrolysis temperature may be between about 600° and about 650° C. The pyrolysis temperature is typically reached by a process in which the temperature is slowly ramped up. For example, when using a pyrolysis temperature of 650° C., the pyrolysis temperature may be achieved by increasing the temperature from 50° C. to 250° C. at a ramp rate of 13.3° C/min, increasing the temperature from 250° C. to 635° C. at a ramp rate of 3.85° C./min, and increasing the temperature from 635° C. to 650° C. at a ramp rate of 0.25° C./min. Once the pyrolysis temperature is reached, the fibers are heated at the pyrolysis temperature for a soak time, which may be a number of hours.

The polymer precursor fibers may also be bundled and pyrolyzed as a bundle in order produce a large amount of modified CMS hollow fiber membranes in a single pyrolysis run. Although pyrolysis will generally be referred to in terms of pyrolysis of a precursor fiber, it should be understood that any description of pyrolysis used herein is meant to include pyrolysis of precursor fibers that are bundled as well as those that are non-bundled.

### Precursor Fibers

The asymmetric polymer precursor fiber may comprise any polymeric material that, after undergoing pyrolysis, produces a CMS membrane that permits passage of the desired gases to be separated, for example carbon dioxide and natural gas, and in which at least one of the desired gases permeates through the CMS fiber at different diffusion rate than other components.

For instance, the polymer may be any rigid, glassy polymer (at room temperature) as opposed to a rubbery polymer or a flexible glassy polymer. Glassy polymers are differentiated from rubbery polymers by the rate of segmental movement of polymer chains. Polymers in the glassy state do not have the rapid molecular motions that permit rubbery polymers their liquid-like nature and their ability to adjust segmental configurations rapidly over large distances (>0.5 nm). Glassy polymers exist in a non- equilibrium state with entangled molecular chains with immobile molecular backbones in frozen conformations. The glass transition temperature (Tg) is the dividing point between the rubbery or glassy state. Above the Tg, the polymer exists in the rubbery state; below the Tg, the polymer exists in the glassy state. Generally, glassy polymers provide a selective environment for gas diffusion and are favored for gas separation applications. Rigid, glassy polymers describe polymers with rigid polymer chain backbones that have limited intramolecular rotational mobility and are characterized by having high glass transition temperatures. Preferred rigid, glassy polymer precursors have a glass transition temperature of at least 200° C.

In rigid, glassy polymers, the diffusion coefficient tends to control selectivity, and glassy membranes tend to be selective in favor of small, low-boiling molecules. For example, preferred membranes may be made from rigid, glassy polymer materials that will pass carbon dioxide, hydrogen sulfide and nitrogen preferentially over methane and other light hydrocarbons. Such polymers are well known in the art and include polyimides, polysulfones and cellulosic polymers.

The polyimides are preferred polymers precursor materials. Suitable polyimides include, for example, Ultem® 1000, Matrimid® 5218, P84, Torlon®, 6FDA-DAM, 6FDA-DAM-DABA, 6FDA-DETDA-DABA, 6FDA/BPDA- DAM, 6FDA-6FpDA, and 6FDA-IPDA.

The polyimide commercially sold as Matrimid® 5218 is a thermoplastic polyimide based on a specialty diamine, 5(6)-amino-l-(4′ aminophenyl)-1,3,-trimethylindane. Its structure is:

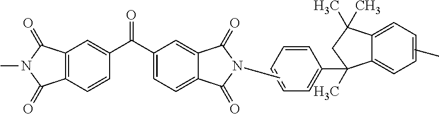

The Matrimid® 5218 polymers used in the Examples were obtained from Huntsman International LLC. 6FDA/BPDA- DAM is a polymer made up of 2,4,6-Trimethyl-1,3-phenylene diamine (DAM), 3,3,4,4-biphenyl tetracarboxylic dianhydride (BPDA), and 5,5-[2,2,2-trifluoro-1-(trifluoromethyl)ethylidene]bis-1,3-isobenzofurandione (6FDA), and having the structure:

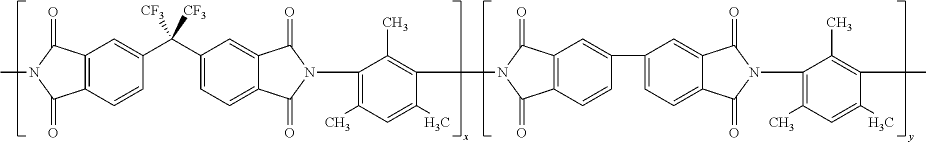

To obtain the above mentioned polymers one can use available sources or synthesize them. For example, such a polymer is described in U.S. Pat. No. 5,234,471, the contents of which are hereby incorporated by reference.

Although polyimide polymers are used in the examples, it is understood that the polyimides are merely examples of rigid, glassy polymers. Accordingly, the preparation of CMS membranes from the polyimides used in the examples is exemplary and representative of the preparation of CMS membranes from rigid, glassy polymers as a class of materials. Similarly, the use of CMS membranes prepared from polyimide precursors for the separation of gases, as demonstrated in the examples, is exemplary and representative of the gas separation performance of CMS membranes prepared from rigid, glassy polymers as a class of materials.

Examples of other suitable precursor polymers include polysulfones; poly(styrenes), including styrene-containing copolymers such as acrylonitrilestyrene copolymers, styrene-butadiene copolymers and styrene- vinylbenzylhalide copolymers: polycarbonates; cellulosic polymers, such as cellulose acetate- butyrate, cellulose propionate, ethyl cellulose, methyl cellulose, nitrocellulose, etc.; poly-amides and polyimides, including aryl polyamides and aryl polyimides; polyethers; polyetherimides; polyetherketones; poly(arylene oxides) such as poly(phenylene oxide) and poly(xylene oxide); poly(esteramide-diisocyanate); polyurethanes; polyesters (including polyarylates), such as poly(ethylene terephthalate), poly(alkyl methacrylates), poly(acrylates), poly(phenylene terephthalate), etc.; polypyrrolones; polysulfides; polymers from monomers having alpha- olefinic unsaturation other than mentioned above such as poly(ethylene), poly(propylene), poly(butene-1), poly(4-methyl pentene-1), polyvinyls, e.g., poly(vinyl chloride), poly(vinyl fluoride), poly(vinylidene chloride), poly(vinylidene fluoride), poly(vinyl alcohol), poly(vinyl esters) such as poly(vinyl acetate) and poly(vinyl propionate), poly(vinyl pyridines), poly(vinyl pyrrolidones), poly(vinyl ethers), poly(vinyl ketones), poly(vinyl aldehydes) such as poly(vinyl formal) and poly(vinyl butyral), poly(vinyl amides), poly(vinyl amines), poly(vinyl urethanes), poly(vinyl ureas), poly(vinyl phosphates), and poly(vinyl sulfates); polyallyls; poly(benzobenzimidazole); polyhydrazides; polyoxadiazoles; polytriazoles; poly(benzimidazole); polycarbodiimides; polyphosphazines; etc., and interpolymers, including block interpolymers containing repeating units from the above such as terpolymers of acrylonitrile-vinyl bromide- sodium salt of para-sulfophenylmethallyl ethers; and grafts and blends containing any of the foregoing. Typical substituents providing substituted polymers include halogens such as fluorine, chlorine and bromine; hydroxyl groups; lower alkyl groups; lower alkoxy groups; monocyclic aryl; lower acyl groups and the like. Other examples of precursor materials may include polymers with intrinsic microporosity (e.g. those disclosed in U.S. Pat. App. Pub. No. 20150165383), thermally-rearranged polymers (e.g. those disclosed in U.S. Pat. App. Pub. No. 20120329958), and mixed-matrix materials (e.g. those disclosed in U.S. Pat. App. Pub. No. 20170189866 A1).

The asymmetric polymer precursor fiber may be a composite structure comprising a first polymer material supported on a porous second polymer material. Composite structures may be formed by using more than one polymer material as the dope during the asymmetric hollow fiber spinning process.

### Formation of CMS Fiber Having a Thin Separation Layer

Embodiments of the present disclosure are directed to methods of preparing an asymmetric CMS hollow fiber membrane having a thin separation layer, also referred to as the skin or the outer skin layer. In some embodiments, for example, the carbon molecular sieve hollow fiber membrane may comprise a porous substrate layer and an outer skin layer having a thickness of 3 microns or less, alternatively 2 microns or less, alternatively 1.5 microns or less, alternatively 1 micron or less.

The method involves preparing a hollow polymer fiber, also referred to as a precursor fiber, having a core layer and a sheath layer, and then pyrolyzing the precursor fiber to prepare a CMS hollow fiber membrane. Preparation of the precursor fiber may involve using a two-layer dope composition comprising a core dope and a sheath dope when spinning the precursor polymer fiber. For instance, one may coextrude a two-layer dope composition and a bore fluid through a spinneret into an air gap, such as during a dry-jet step. The resulting fiber may then be immersed in an aqueous quench bath, such as during a wet-quench step. After the wet phase separation occurs, the dual-phase precursor fibers may be collected by a take-up drum and subjected to a solvent exchange process. In some embodiments, the precursor fibers may also be treated with one or more modifying agents that are known to reduce substructure collapse (which may be introduced into the solvent exchange process, such as is described in U.S. patent application Ser. No. 14/501,884 (published as US 2015/0094445 A1), the entirety of which is incorporated by reference).

The two-layer dope composition and spinning parameters may be controlled so that the sheath layer has a thickness of 3 microns or less, alternatively 2 microns or less, alternatively 1.5 microns or less, alternatively 1 micron or less. For example, the sheath layer thickness may be controlled by tuning the dope flow rate, the drum take-up speed, or a combination thereof. In some embodiments, the thickness of the outer skin layer of the CMS fiber may be substantially the same as the thickness of the sheath layer of the precursor fiber. Accordingly, careful control over the thickness of the sheath layer of the precursor fiber may be used to prepare CMS hollow fiber membranes having tuned separation properties.

The core layer may comprise one or more pore-forming chemicals. For instance, one or more pore-forming chemicals may be introduced into the core dope prior to spinning of the precursor fiber. In some embodiments, the pore-forming chemicals may be present in the core dope, and hence in the core layer of the precursor fiber, at a concentration between about 0.5 wt. % and about 20 wt. %, alternatively between about 1 wt. % and about 15 wt. %., alternatively between about 2 wt. % and about 12 wt. %, alternatively between about 2 wt. % and about 10 wt. %, alternatively between about 3 wt. % and about 9 wt. %. The one or more pore-forming chemicals may comprise polyvinylpyrrolidone (PVP).

In some embodiments, the core layer and the sheath layer comprise the same polymer or substantially the same polymer. For example, the core layer and the sheath layer may each comprise a polyimide or a combination of polyimides, including for example any of the polyimides described above. Alternatively, the core layer and the sheath layer may each comprise one or more of the other suitable precursor polymers described above.

Where the two layers of the dual layer precursor fiber comprise the same polymer or combination of polymers, the gas separation properties of the resulting CMS fiber may easily be compared against a single layer CMS fiber prepared from the same polymer composition under the same conditions (e.g. the same post-spinning processing, the same pyrolysis conditions, etc.). In some embodiments, the CMS hollow fiber membrane prepared from a dual-layer precursor fiber may comprise a CO₂ permeance (measured at 100 psia and 35° C.) at least 2 times greater than the CO₂ permeance of the CMS hollow fiber membrane prepared from a single-layer precursor fiber under the same conditions, alternatively at least 3 times greater, alternatively at least 4 times greater, alternatively at least 5 times greater.

In some embodiments, the core layer and the sheath layer comprise different polymers. For example, one or both of the core layer and the sheath layer may comprise a polyimide or a combination of polyimides, including for example any of the polyimides described above. Alternatively, one or both of the core layer and the sheath layer may comprise one or more of the other suitable precursor polymers described above. In some embodiments, the core layer may comprise one or more polyimides and the sheath layer may comprise the combination of one or more polyimides and one or more polyamides. The one or more polyamides may be introduced into the sheath layer through the pre-pyro hybridization process described herein.

Pyrolysis of the dual-layer precursor fiber may be performed as described above and results in an asymmetric CMS hollow fiber membrane having an ultrathin separation (i.e. outer skin) layer. Specifically, during pyrolysis the sheath layer of the precursor fiber is transformed into the separation layer of the CMS fiber. The core layer of the precursor fiber also undergoes densification, but may avoid structural collapse, especially where the precursor fiber has been treated with a modifying agent known to reduce substructure collapse. Accordingly, the core layer of the precursor fiber forms a porous substrate having desirable gas permeance/permeability properties. Moreover, in contrast to conventional asymmetric CMS hollow fiber membranes, there may be a sharp boundary between the porous substrate layer and the outer separation layer, as seen for example in FIG. 5.

### Pre-Pyro Hybridization

Embodiments of the present disclosure are directed to a method for eliminating possible defects or packing irregularities in the skin layer of an asymmetric CMS hollow fiber membrane. The method involves treating a hollow polymer fiber, i.e. a precursor fiber, to introduce an in situ polymerized polymeric material onto an outer surface of the hollow polymer fiber, into interstitial defects of the hollow polymer fiber, or both, thereby forming a hybrid outer layer, and then pyrolyzing the treated hollow polymer fiber to prepare a carbon molecular sieve hollow fiber membrane.

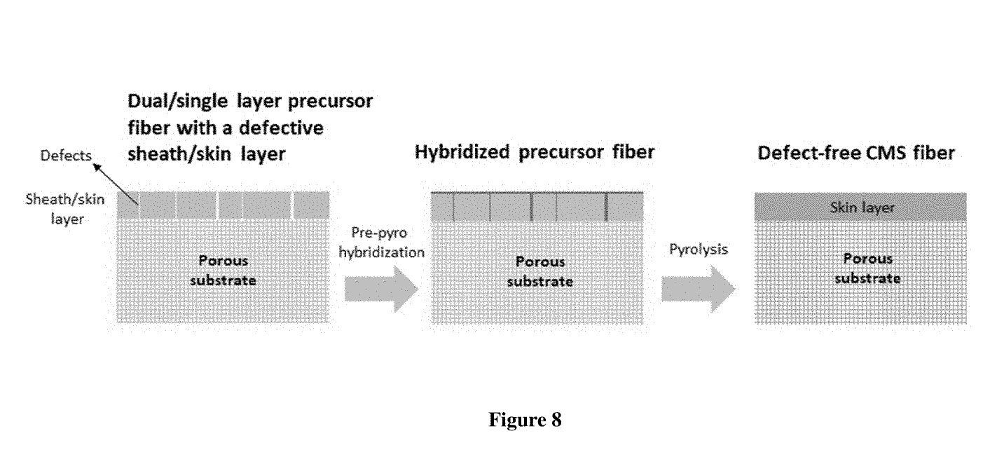

In some embodiments, the precursor fiber may be a dual-layer precursor fiber, such as one prepared by the process described above. In such embodiments, the in situ polymerized polymeric material may be introduced onto an outer surface of the sheath layer of the precursor fiber, into interstitial defects in the sheath layer of the precursor fiber, or both, thereby forming a precursor fiber having a hybridized sheath layer. An example of this process is shown schematically in FIG. 8. In other embodiments, the precursor fiber may be an asymmetric single-layer precursor fiber having a defective skin layer. In such embodiments, the in situ polymerized polymeric material may be introduced onto an outer surface of the skin layer of the precursor fiber, into interstitial defects in the skin layer of the precursor fiber, or both, thereby forming a precursor fiber having a hybridized skin layer. An example of this process is also shown schematically in FIG. 8.

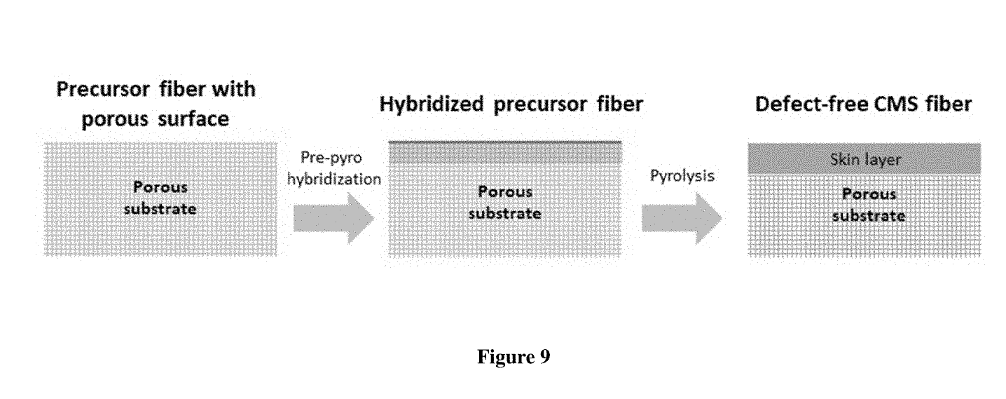

In yet other embodiments, the precursor fiber may be a single-layer precursor fiber having a porous surface and being without a typical skin layer. In such embodiments, the in situ polymerized polymeric material may be introduced onto an outer surface of the porous surface of the precursor fiber, into the pores in the porous surface of the precursor fiber, or both, thereby forming a precursor fiber having a hybridized outer layer. An example of this process is shown schematically in FIG. 9.

Treating the hollow polymer fiber to introduce an in situ polymerized polymeric material may involve at least a two-step process. In a first step, the hollow polymer fiber may be contacted with a first solution comprising a first monomer material. In some embodiments the first solution may comprise between about 0.0001 wt. % and about 1 wt. % of the first monomer, alternatively between about 0.001 wt. % and about 0.1 wt. %, in an appropriate solvent. The contacting may be performed by soaking the hollow polymer fiber in the first solution. In some embodiments, the soaking may be performed for one hour or less, alternatively thirty minutes or less. The soaking time, the concentration of the monomer in the solution, or both may be selected to introduce a desired amount of the first monomer (and hence a desired amount of in situ polymerized polymeric material) into the hollow polymer fiber.

In a second step, the hollow polymer fiber may be contacted with a second solution comprising a second monomer material, such that the second monomer material reacts with the first monomer material present on and/or within the hollow polymer fiber resulting from the first step to form an in situ polymerized polymeric material. In some embodiments the second solution may comprise between about 0.0001 wt. % and about 1 wt. % of the second monomer, alternatively between about 0.001 wt. % and about 0.1 wt. %, in an appropriate solvent. The contacting may be performed by soaking the hollow polymer fiber in the second solution. In some embodiments, the soaking may be performed for one hour or less, alternatively thirty minutes or less. The soaking time, the concentration of the monomer in the solution, or both may be selected to introduce a desired amount of the second monomer (and hence a desired amount of in situ polymerized polymeric material) into the hollow polymer fiber. After the second step, the resulting hollow polymer fiber may be dried prior to pyrolysis, such as to remove any unreacted monomers.

In some embodiments, the in situ polymerized polymeric material may comprise polyamide, polyimide, or polyamide-imide. A polyamide may be formed in situ, for example, by the reaction between a multi-functional amine and a multi-functional acyl halide. For instance, the first monomer may comprise a diamine, a triamine, etc. The second monomer may comprise a di-acyl halide, a tri-acyl halide, a tetra-acryl halide, etc. The halide may be a chloride, a bromide, a fluoride, etc. For instance, in some embodiments, the second monomer may comprise a di-acyl fluoride or a tri-acyl chloride. For example, the first monomer may comprise 2,5-diethyl-6-methyl-1,3-diamino benzene and the second monomer may comprise trimesoyl chloride. As another example, the in situ polymerized polyamide material can be formed by the reaction between a diamine and a tetra acryl chloride. A polyimide can be formed in situ by introduction of a poly (amic acid) followed by imidization of the poly (amic acid), such as by thermal or chemical imidization. A polyamide-imide can be formed in situ by introduction of an appropriate amino aromatic acid followed by thermal or chemical imidization.

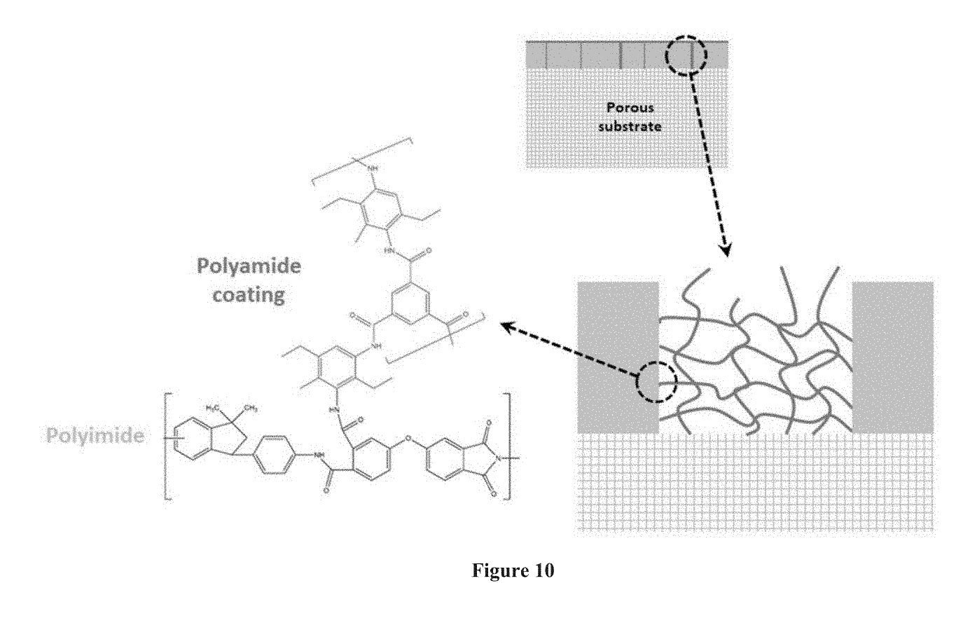

A pre-pyrolysis hybridization process involving the introduction of an example of an in situ polymerized polyamide material is illustrated in FIG. 10. As the precursor hollow fiber is sequentially soaked in diamine and tri-acyl chloride solutions, polyamides are formed resulting in a polyimide-polyamide hybrid skin layer. Specifically, as illustrated in FIG. 10 for example, the polyamides may form both on surface of the polyimide hollow fiber and inside small interstitial defects of the skin layer (or the sheath layer in the case of a dual-layer precursor fiber). The polyamide polymer chains may physically fill in the defects. As illustrated in FIG. 10, the amine groups on the polyamide may also react with the imide group of the polyimide hollow fiber to improve adhesion of the polyamide polymer chains to the precursor hollow fiber. Polyamides are rigid polymers with strong inter-chain hydrogen bonding. As the hybridized precursor hollow fiber is pyrolyzed, the polyimide-polyamide hybrid skin layer is transformed into an integral, dense CMS skin layer. The CMS hollow fibers derived from polyamide-hybridized precursor hollow fibers have excellent mechanical strength and attractive separation performance.

The pre-pyro hybridization treatment may increase the selectivity of the resulting asymmetric CMS hollow fiber membrane, for instance by eliminating possible defects or packing irregularities. In some embodiments, for example, the CMS hollow fiber membrane may comprise a CO₂/CH₄ selectivity (measured at 100 psia and 35° C.) that is at least double the CO₂/CH₄ selectivity of a CMS hollow fiber membrane prepared under the same conditions but without the pre-pyro hybridization treatment step. While pre-pyro hybridization is able to increase selectivities of CMS hollow fiber membranes, it also adds mass transfer resistance to gas permeation. Accordingly, pre-pyro hybridization may cause the gas permeance/permeability properties of the resulting CMS hollow fiber membranes to be reduced. For example, the CO₂ permeances of the CMS hollow fibers may be reduced with increasing polyamide concentrations

### CMS Membranes and Gas Separation

Embodiments of the present disclosure are also directed to the asymmetric carbon molecular sieve hollow fiber membranes prepared by any of the methods disclosed herein. Embodiments of the present disclosure are also directed to the use of the asymmetric CMS hollow fiber membranes disclosed herein in processes for separating at least a first gas component and a second gas component.

In some embodiments, the process may comprise providing a carbon molecular sieve membrane prepared by any of the processes disclosed herein and contacting a gas stream comprising at least a first gas component and a second gas component with the carbon molecular sieve membrane to produce i. a retentate stream having a reduced concentration of the first gas component, and ii. a permeate stream having an increased concentration of the first gas component. In some embodiments, the first gas component may be CO₂, H₂S, or a mixture thereof and the second gas component may be CH₄. For instance, in some embodiments, the process may comprise separating acid gas components from a natural gas stream by providing a carbon molecular sieve membrane prepared by any of the process disclosed herein and contacting a natural gas stream containing one or more acid gas components with the carbon molecular sieve membrane to produce i. a retentate stream having a reduced concentration of acid gas components, and ii. a permeate stream having an increased concentration of acid gas components. Other gas pairings that can be separated using the CMS hollow fiber membranes disclosed herein include CO₂ and N₂, O₂ and N₂, N₂ and CH₄, He and CH₄, H₂ and CH₄, H₂ and C₂H₄, ethylene and ethane, propylene and propane, ethylene/propylene and ethane/propane, n-butane and iso-butane, iso-butylene and iso-butane, butadiene from a mixture of C₄s, a first pentane isomer from a second pentane isomer, a first hexane isomer from a second hexane isomer, a first xylene isomer from a second xylene isomer, and the like.

### EXAMPLES 1 THROUGH 7

### Materials:

Matrimid® 5218 polyimide (T_{g}=305-310° C.) was used as the precursor polymer in these examples. The polyvinylpyrrolidone (PVP) was obtained from Sigma-Aldrich with Mw ˜1,300,000. The chemical structure of PVP is as follows:

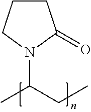

The precursor hollow fiber skin layer was hybridized with polyamides by reacting 2,5-diethyl-6-methyl-1,3-diamino benzene (DETDA) and trimesoyl chloride (TMC). The chemical structure of the crosslinked polyamide is shown below:

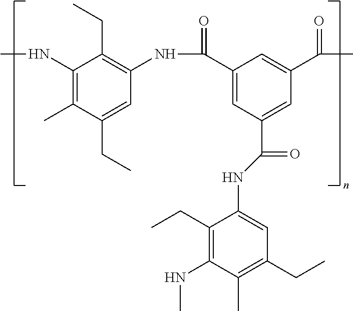

### Formation of the Comparative Precursor Hollow Fibers (Precursor A):

Single-layer precursor hollow fiber membranes were spun using the “dry-jet/wet-quench” technique. Spinning dope compositions and spinning parameters are listed in Tables 1 and 2. The bore fluid comprised 96 wt % NMP and 4 wt % water. The as-spun hollow fiber membranes experienced sequential soaking in water (3 days), pure methanol (60 mins), and pure hexane (60 mins). The fibers were then dried under vacuum at 75° C. for 2 hours.

TABLE 1
Spinning dope compositions of precursor A

| Component | wt % |
|---|---|
| Matrimid ® | 26.2 |
| N-Methyl-pyrrolidone | 53 |
| Tetrahydrofuran | 5.9 |
| Ethanol | 14.9 |

TABLE 2
Spinning parameters of precursor A

| Spinning parameter | Range |
|---|---|
| Dope temperature (° C.) | 60 |
| Quench bath temperature (° C.) | 50 |
| Dope flow rate (ml/h) | 180 |
| Bore fluid flow rate (ml/h) | 60 |
| Air gap (cm) | 10 |
| Fiber take-up rate (m/min) | 20 |

Formation of Dual-Layer Precursor Hollow Fibers with Engineered Morphology (Precursor B and C):

Dual-layer precursor hollow fiber membranes with engineered morphology were spun using the “dry-jet/wet-quench” technique. Two spinning dopes (sheath dope and core dope) with different compositions were used to form the dual-layer hollow fibers.

Pore formation in hollow fiber substrate can be assisted by adding pore formers into core spinning dope. Examples of pore forming chemicals include lithium nitrate (LiNO₃) and polyvinylpyrrolidone (PVP). LiNO₃, however, is not effective to resist substrate collapse during pyrolysis. In the current invention, PVP was added to the core spinning dope to increase core layer porosity and pore size. The Mw of PVP was 1,300,000 and the weight percentage in the spinning dope was about 6 wt %. Using the present disclosure, one skilled in the art can combine different Mw and weight percentages to obtain a desirable porosity. In parallel, polymer concentration was reduced in the core dope to further assist pore formation. Spinning dope compositions and spinning parameters for precursors B and C are listed in Table 3 and 4. It should be noted that the core spinning dope does not comprise volatile components (tetrahydrofuran and ethanol). A small amount of LiNO₃ was added to the core spinning dope to tune the rate of phase separation. The bore fluid comprised 88 wt % NMP and 12 wt % water.

TABLE 3
Spinning dope compositions of precursor B and C.

| Component | wt % (Sheath dope) | wt % (Core dope) |
|---|---|---|
| Matrimid ® | 26.2 | 18 |
| N-Methyl-pyrrolidone | 53 | 75 |
| Tetrahydrofuran | 5.9 | N/A |
| Ethanol | 14.9 | N/A |
| LiNO₃ | N/A | 1 |
| PVP | N/A | 6 |

TABLE 4
Spinning parameters of precursor B and C.

| Spinning parameter | Precursor B | Precursor C |
|---|---|---|
| Dope temperature (° C.) | 60 | 60 |
| Quench bath temperature (° C.) | 50 | 50 |
| Sheath dope flow rate (ml/h) | 5 | 3 |
| Core dope flow rate (ml/h) | 150 | 150 |
| Bore fluid flow rate (ml/h) | 55 | 55 |
| Air gap (cm) | 10 | 10 |
| Fiber take-up rate (m/min) | 20 | 20 |

Treating Precursor Hollow Fiber Membranes with Modifying Agent (VTMS-Treatment):

Each of the precursor fibers were first soaked in a 10% vinyltrimethoxysilane (VTMS)/hexane solution for 24 hours at room temperature. The precursor hollow fibers were then brought into contact with water vapor-saturated air for another 12 hours at room temperature. More details regarding the VTMS treatments are described in U.S. Pat. No. 9,211,504 B2 and U.S. patent application Ser. No. 14/501,884 (published as US 2015/0094445 A1), the entireties of which are incorporated herein by reference.

### Pre-Pyro Hybridization:

Prior to pyrolysis, the polyimide skin layer of precursor hollow fiber B and C was hybridized with polyamides by sequential soaking in diamine/hexane and tri-acyl chloride/hexane monomer solutions. The fibers were first soaked in a dilute 2,5-diethyl-6-methyl-1,3-diamino benzene (DETDA)/hexane solution for 30 mins. After the DETDA/hexane solution was drained, the precursor hollow fibers were then soaked in a dilute trimesoyl chloride (TMC)/hexane solution for another 30 mins. After the TMC/hexane solution was drained, the fibers were dried in a vacuum oven at 150° C. for 12 hours. Since the hybridization was applied to the precursor hollow fiber before pyrolysis, the technique may be known as “pre-pyro hybridization”. The monomer concentrations for different hybridization conditions are shown in Table 5.

TABLE 5
Monomer concentrations for the pre-pyro hybridization.

| Hybridization conditions | DETDA wt % | TMC wt % |
|---|---|---|
| 0.1% DETDA/TMC | 0.1 | 0.1 |
| 0.001% DETDA/TMC | 0.001 | 0.001 |

### Formation of CMS Hollow Fiber Membranes:

The hybridized precursor hollow fibers were placed on a wired stainless steel mesh support in a quartz tube, and then loaded into a pyrolysis furnace (Thermocraft, Inc., model 23-24-1ZH, Winston-Salem, N.C., USA). The entire system was purged with ultra-high purity (UHP) argon for at least 12 hours until 0₂ level in the system dropped below 1 ppm. Pyrolysis was performed using the heating protocol below under continuous purge of UHP argon (200 cc/min). After the heating protocol was completed, the furnace was naturally cooled down to room temperature.

### Heating Protocol:

1) 50° C. to 250° C. (13.3° C./min)
2) 250° C. to T_{final}−15 (3.85° C./min)
3) T_{final}−15 to T_{final} (0.25° C./min)
4) Thermal soak at T_{final}−15 for 120 min
5) Cool down naturally
T_{final}=550 and 650° C.

### Results:

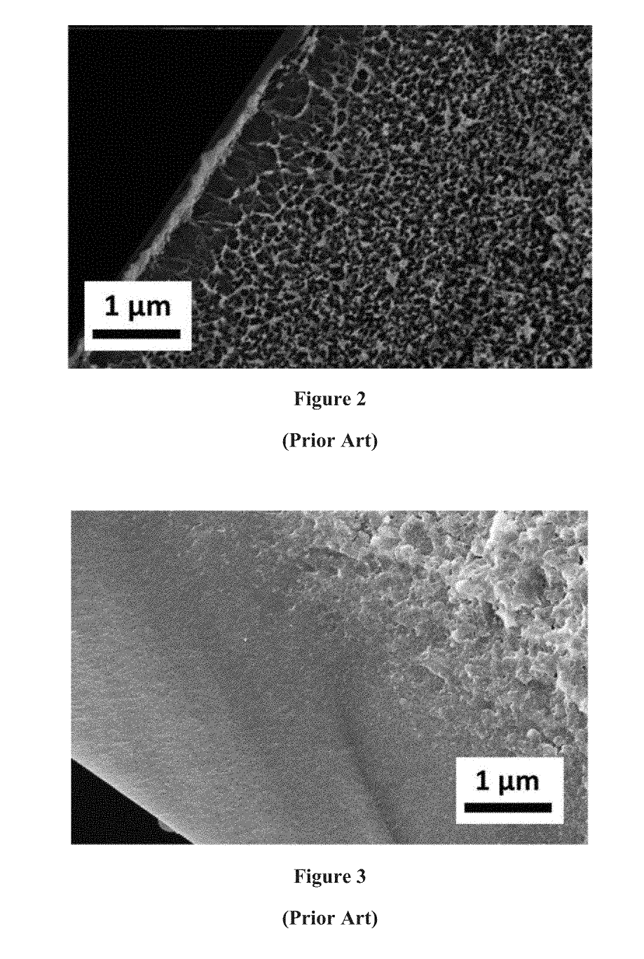

Morphology of the CMS hollow fibers were characterized with a LEO 1530 field-emission scanning microscope microscopy (SEM). FIG. 2 shows the morphology of precursor A, which has a skin layer of about 1.0 μm. FIG. 3 shows the CMS hollow fiber membrane pyrolyzed from precursor A. The separation layer of the CMS hollow fiber membrane was about 5 μm. The substrate below the skin layer of precursor A was partially densified during pyrolysis, even with VTMS treatment. Accordingly, the skin layer of the CMS hollow fiber membrane was much thicker than the precursor fiber skin layer.

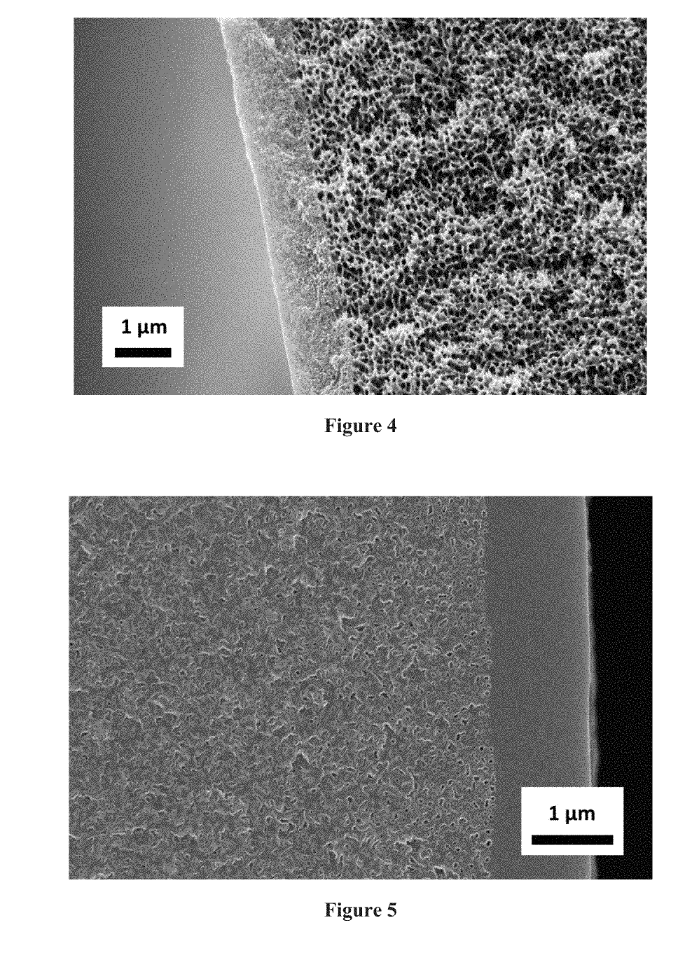

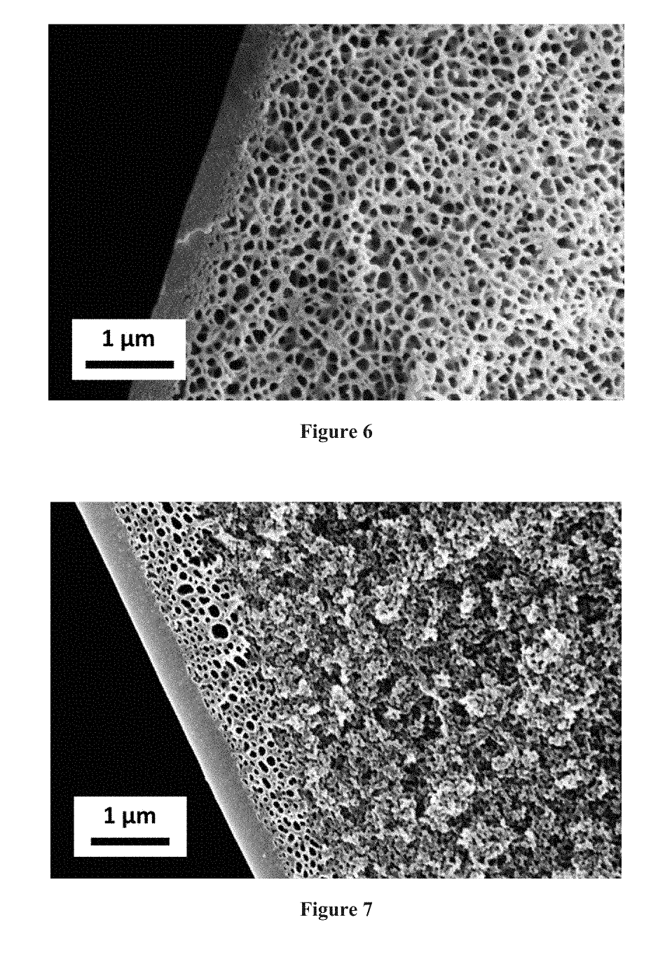

FIG. 4 shows the morphology of precursor B, which showed a clear boundary between sheath layer and core layer. Similar to precursor A, precursor B had a skin layer of about 1.0 μm. However, precursor B clearly had more open substrate with larger pores and higher interconnectivity. This was due to the addition of PVP to the core spinning dope. Such morphological difference resulted in substantially different separation layer thickness in the resulting CMS hollow fiber membranes. As precursor B was pyrolyzed, its sheath layer was transformed into the separation layer of the CMS hollow fibers. With higher porosity and increased pore size, the substrate underneath precursor B′s sheath layer sustained densification during pyrolysis without structural collapse. As shown by FIG. 5, the CMS hollow fiber membrane pyrolyzed from precursor B had a much thinner separation layer of about 1.0 μm. In contrast to FIG. 3 where the transition from separation layer to substrate is blurry, a sharp interface can be seen in FIG. 5.

Similar to Precursor B, a clear boundary was seen between the sheath layer and core layer in precursor C. The sheath layer of precursor C was about 400 nm, which was thinner than that of precursor B. This is enabled by a reduced sheath dope flow rate (3 vs 5 cc/hr) as precursor C was spun. The thinner sheath layer of precursor C contributed to a thinner skin layer in the CMS hollow fiber membrane. As shown in FIG. 7, the skin layer of the CMS hollow fiber pyrolyzed from precursor C was about 400 nm. By comparing FIGS. 5 and 7, it is clear that CMS hollow fiber skin layer thickness can be controlled by control of the spinning conditions of the dual-layer precursor hollow fibers (e.g. by control of the sheath dope flow rate).

Separation performance of CMS hollow fibers membranes were characterized with 50%/50% CO₂/CH₄ mixture permeation at 35° C. and 100 psia upstream pressure (downstream was at 1 atm). The feed mixture was introduced to the fiber shell side and permeate was withdrawn from the fiber bore side. Permeate flow rate was measured using a bubble flow meter (10 ml) and compositions were analyzed using a Varian-430 gas chromatograph (GC). The stage-cut, which is the percentage of feed mixture that permeates through the membrane, was kept less than 1% to avoid concentration polarization.

CO₂/CH₄ separation performance of ultrathin CMS hollow fiber membranes pyrolyzed from precursor B (at 550 and 650° C.) and precursor C (at 550° C.) under UHP Argon are summarized in Table 6. The data are compared with CMS hollow fiber membranes pyrolyzed from precursor A.

TABLE 6
CO₂/CH₄ equimolar mixture permeation data (100 psia, 35°
C.) of Matrimid ®-derived CMS hollow fiber membranes
at different pyrolysis temperatures. All precursors were
treated with 10% VTMS/hexane solution prior to pyrolysis.

| | | Skin | P(CO₂)/ | α(CO₂/ |
|---|---|---|---|---|
| | CMS hollow fiber membrane | (μm) | GPU | CH₄) |
| 550° C. | Pyrolyzed using precursor A | ~5 | ~110 | ~13 |
| pyrolysis | Pyrolyzed using precursor B | ~1 | 1830 | 9 |
| | w/o pre-pyro hybridization | | | |
| | Pyrolyzed using precursor B | ~1 | 1177 ± 320 | 37 ± 14 |
| | treated with pre-pyro | | | |
| | hybridization (0.1 wt % | | | |
| | DETDA/TMC) | | | |
| | Pyrolyzed using precursor B | ~1 | 1452 | 18 |
| | treated with pre-pyro | | | |
| | hybridization (0.001 wt % | | | |
| | DETDA/TMC) | | | |
| | Pyrolyzed using precursor C | ~0.35 | 1310 ± 25 | 35 ± 2 |
| | treated with pre-pyro | | | |
| | hybridization (0.1 wt % | | | |
| | DETDA/TMC) | | | |
| 650° C. | Pyrolyzed using precursor A | ~5 | ~35 | ~90 |
| pyrolysis | Pyrolyzed using precursor B | ~1 | 290 | 29 |
| | w/o pre-pyro hybridization | | | |
| | Pyrolyzed using precursor B | ~1 | 154 | 70 |
| | treated with pre-pyro | | | |
| | hybridization (0.1 wt % | | | |
| | DETDA/TMC) | | | |
| | Pyrolyzed using precursor B | ~1 | 205 | 67 |
| | treated with pre-pyro | | | |
| | hybridization (0.01 wt % | | | |
| | DETDA/TMC) | | | |

After the pre-pyro hybridization, the ultrathin CMS hollow fiber membranes showed more attractive CO₂/CH₄ separation factors. The pre-pyro hybridization reduced CO₂ permeance of the ultrathin CMS hollow fiber; however, the permeances are still substantially higher than CMS hollow fiber membranes pyrolyzed from precursor A. For the ultrathin CMS hollow fiber membranes pyrolyzed from Precursor B at 550° C. (pre-pyro hybridization with 0.1 wt % DETDA/TMC), the CO₂ permeance was 1177 GPU, which was ˜970% higher than CMS hollow fiber membranes pyrolyzed from precursor A. In the meantime, the ultrathin CMS hollow fiber shows very attractive CO₂/CH₄ separation factor of ˜37. As the pre-pyro hybridization monomer concentrations was reduced to 0.001 wt %, the CO₂ permeance was increased to 1452 GPU. While the CO₂/CH₄ separation factor was further reduced, the value (18) was still attractive.

In some embodiments, when pyrolysis is performed at about 550° C., the CMS hollow fiber membrane resulting from pyrolysis of a dual-layer precursor fiber that has been treated by pre-pyro hybridization may comprise a CO₂/CH₄ selectivity of at least 15 and a CO₂ permeance of at least 1100 GPU, when measured at 100 psia and 35° C. In some embodiments, when pyrolysis is performed at about 650° C., the CMS hollow fiber membrane resulting from pyrolysis of a dual-layer precursor fiber that has been treated by pre-pyro hybridization may comprise a CO₂/CH₄ selectivity of at least 65 and a CO₂ permeance of at least 150, when measured at 100 psia and 35° C.

Moreover, one may control the thickness of the sheath layer in the precursor fiber, the amount of in-situ polymerized polymeric material introduced into the precursor fiber, or both, in order to provide a CMS hollow fiber membrane having a desired combination of gas permeance and selectivity properties for a particular application. For example, as shown in Table 6, with reduced skin layer thickness (400 nm vs 1 μm), the ultrathin CMS hollow fiber membrane pyrolyzed from precursor C showed increased CO₂ permeance (1310 GPU) while maintaining highly attractive CO₂/CH₄ selectivity (˜535).

### EXAMPLES 8 THROUGH 11

Additional experimentation was performed by preparing dual-layer precursor hollow fiber membranes having a sheath layer made from a different polymer than that of the core layer. In particular, Matrimid® 5218 polyimide was used as the core dope and 6FDA/BPDA-DAM was used as the sheath dope. The spinning dope compositions for precursors D through G are provided in Table 7 and the spinning parameters for precursors D through G are provided in Table 8.

TABLE 7
Spinning dope compositions of precursors D through G.

| Component | wt % (Sheath dope) | wt % (Core dope) |
|---|---|---|
| Matrimid ® | N/A | 18 |
| 6FDA:BPDA-DAM | 20 | N/A |
| N-Methyl-pyrrolidone | 47.5 | 75 |
| Tetrahydrofuran | 10.0 | N/A |
| Ethanol | 16.0 | N/A |
| LiNO₃ | 6.5 | 1 |
| PVP | N/A | 6 |

TABLE 8
Spinning parameters for precursors D through G.

| | Precur- | Precur- | Precur- | Precur- |
|---|---|---|---|---|
| Spinning parameter | sor D | sor E | sor F | sor G |
| Dope temperature (° C.) | 60 | 60 | 60 | 60 |
| Quench bath temp (° C.) | 50 | 50 | 50 | 50 |
| Sheath dope flow rate (ml/h) | 5 | 5 | 3 | 5 |
| Core dope flow rate (ml/h) | 300 | 600 | 155 | 155 |
| Bore fluid flow rate (ml/h) | 100 | 200 | 55 | 55 |
| Air gap (cm) | 10 | 10 | 10 | 10 |
| Fiber take-up rate (m/min) | 20 | 20 | 30 | 30 |

Treating Precursor Hollow Fiber Membranes with Modifying Agent (VTMS-Treatment):

Each of precursor fibers D through G were first soaked in a 10% vinyltrimethoxysilane (VTMS)/hexane solution for 24 hours at room temperature. The precursor hollow fibers were then brought into contact with water vapor-saturated air for another 12 hours at room temperature.

### Pre-Pyro Hybridization:

Prior to pyrolysis, the skin layer of precursor hollow fibers D through G was hybridized with polyamides by sequential soaking in diamine/hexane and tri-acyl chloride/hexane monomer solutions. The fibers were first soaked in a dilute (0.1 wt. %) 2,5-diethyl-6-methyl-1,3-diamino benzene (DETDA)/hexane solution for 30 mins. After the DETDA/hexane solution was drained, the precursor hollow fibers were then soaked in a dilute (0.1 wt. %) trimesoyl chloride (TMC)/hexane solution for another 30 mins. After the TMC/hexane solution was drained, the fibers were dried in a vacuum oven at 150° C. for 12 hours. Since the hybridization was applied to the precursor hollow fiber before pyrolysis, the technique may be known as “pre-pyro hybridization”.

### Formation of CMS Hollow Fiber Membranes:

The hybridized precursor hollow fibers were placed on a wired stainless steel mesh support in a quartz tube, and then loaded into a pyrolysis furnace (Thermocraft, Inc., model 23-24-1ZH, Winston-Salem, NC, USA). The entire system was purged with ultra-high purity (UHP) argon for at least 12 hours until 0₂ level in the system dropped below 1 ppm. Pyrolysis was performed using the heating protocol below under continuous purge of UHP argon (200 cc/min). After the heating protocol was completed, the furnace was naturally cooled down to room temperature.

### Heating Protocol:

1) 50° C. to 250° C. (13.3° C./min)
2) 250° C. to T_{final}−15 (3.85° C./min)
3) T_{final}−15 to T_{final} (0.25° C./min)
4) Thermal soak at T_{final}−15 for 120 min
5) Cool down naturally
T_{final}=550° C.

### Results:

Separation performance of the CMS hollow fibers membranes was characterized with pure gas CO₂ and CH₄ permeation at 35° C. and 100 psia upstream pressure (downstream was at 1 atm). Each pure gas (CO₂ and CH₄) was independently introduced to the fiber shell side and permeate was withdrawn from the fiber bore side. Permeate flow rate was measured using a bubble flow meter (10 ml). Selectivity was then calculated based on the permeation data of each gas. The resulting CO₂/CH₄ separation performance of ultrathin CMS hollow fiber membranes pyrolyzed from precursors D, F, and G are summarized in Table 9.

TABLE 9
CO₂/CH₄ single gas permeation data (100 psia, 35°
C.) of CMS hollow fiber membranes derived from Matrimid ® 5218
core/6FDA/BPDA-DAM sheath precursors.

| CMS hollow fiber membrane | P(CO₂)/GPU | α(CO₂/CH₄) |
|---|---|---|
| Pyrolyzed from Precursor D | 3144 ± 517 | 10 ± 2 |
| Pyrolyzed from Precursor F | 1365 ± 115 | 21 ± 6 |
| Pyrolyzed from Precursor G | 2362 ± 108 | 15 ± 2 |

Separation performance of the CMS hollow fibers membranes was also characterized with equimolar 50%/50% CO₂/CH₄ mixture permeation at 35° C. and 100 psia upstream pressure (downstream was at 1 atm). The feed mixture was introduced to the fiber shell side and permeate was withdrawn from the fiber bore side. Permeate flow rate was measured using a bubble flow meter (10 ml) and compositions were analyzed using a Varian-430 gas chromatograph (GC). The stage-cut, which is the percentage of feed mixture that permeates through the membrane, was kept less than 1% to avoid concentration polarization.

CO₂/CH₄ separation performance of ultrathin CMS hollow fiber membranes pyrolyzed from precursors D and E are summarized in Table 10.

TABLE 10
CO₂/CH₄ mixed gas permeation data (100 psia, 35°
C.) of CMS hollow fiber membranes derived from Matrimid ® 5218
core/6FDA/BPDA-DAM sheath precursors.

| CMS hollow fiber membrane | P(CO₂)/GPU | α(CO₂/CH₄) |
|---|---|---|
| Pyrolyzed from Precursor E | 1980 | 30.7 |
| Pyrolyzed from Precursor D | 2070 | 32.3 |

The ultrathin CMS hollow fiber membranes derived from dual layer polymer precursors having a first polymer as the core layer and a second, different, polymer as the sheath layer showed particularly attractive CO₂/CH₄ separation factors. Thus, aspects of the present disclosure may be used to prepare a CMS hollow fiber membrane having customized properties by selection of the polymers used as the core and/or sheath layers, in addition to varying the thickness of the sheath layer, varying the concentrations of the pre-pyro hybridization agents, varying the pyrolysis temperature, and the like.

This disclosure reveals methods to form ultrathin CMS hollow fiber membranes with attractive permeances and selectivities. By using VTMS-treated dual-layer precursor hollow fiber membranes with ultrathin sheath layer and increased substrate porosity, ultrathin CMS hollow fiber membranes were formed by pyrolysis in a standard CMS formation process with sub-micron skin layer. By hybridizing the precursor hollow fibers with polyamide prior to pyrolysis (pre-pyro hybridization), the ultrathin CMS hollow fiber membranes showed substantially increased CO₂ permeance and attractive CO₂/CH₄ separation factors. The ultrathin CMS hollow fiber membranes pyrolyzed at 550° C. had CO₂/CH₄ separation factors of ˜37. Also, the CMS membrane showed CO₂ permeances (1177 GPU) that are substantially higher (˜970%) than CMS hollow fiber membranes pyrolyzed from traditional precursor hollow fibers under identical pyrolysis conditions.

In some embodiments, a CMS hollow fiber membrane resulting from pyrolysis of a dual-layer precursor fiber that has been treated by pre-pyro hybridization may, when measured using an equimolar gas mixture at 100 psia and 35° C., have CO₂ permeance and CO₂/CH₄ selectivity properties of at least 200/15 (written as permeance (GPU)/selectivity), alternatively at least 300/15, alternatively at least 400/15, alternatively at least 500/15, alternatively at least 600/15, alternatively at least 700/15, alternatively at least 800/15, alternatively at least 900/15, alternatively at least 1000/15, alternatively at least 1100/15, alternatively at least 200/20, alternatively at least 300/20, alternatively at least 400/20, alternatively at least 500/20, alternatively at least 600/20, alternatively at least 700/20, alternatively at least 800/20, alternatively at least 900/20, alternatively at least 1000/20, alternatively at least 1100/20, alternatively at least 200/25, alternatively at least 300/25, alternatively at least 400/25, alternatively at least 500/25, alternatively at least 600/25, alternatively at least 700/25, alternatively at least 800/25, alternatively at least 900/25, alternatively at least 1000/25, alternatively at least 1100/25, alternatively at least 200/30, alternatively at least 300/30, alternatively at least 400/30, alternatively at least 500/30, alternatively at least 600/30, alternatively at least 700/30, alternatively at least 800/30, alternatively at least 900/30, alternatively at least 1000/30, alternatively at least 1100/30.

In other embodiments, a CMS hollow fiber membrane resulting from pyrolysis of a dual-layer precursor fiber that has been treated by pre-pyro hybridization may, when measured using an equimolar gas mixture at 100 psia and 35° C., have CO₂ permeance and CO₂/CH₄ selectivity properties of at least 100/50 (written as permeance (GPU)/selectivity), alternatively at least 150/50, alternatively at least 200/50, alternatively at least 250/50, alternatively at least 100/60, alternatively at least 150/60, alternatively at least 200/60, alternatively at least 300/60, alternatively at least 100/65, alternatively at least 150/65, alternatively at least 200/65, alternatively at least 250/65.

In some embodiments, the CMS hollow fiber membrane resulting from pyrolysis of a dual-layer precursor fiber that has been treated by pre-pyro hybridization may have (a) a CO₂/CH₄ selectivity that is at least 75% of the selectivity of a CMS hollow fiber membrane resulting from pyrolysis of a single-layer precursor hollow fiber at the same temperature (when measured at 100 psia and 35° C.), alternatively at least equal to, alternatively at least 1.5 times higher, alternatively at least 1.75 times higher, alternatively at least 2 times higher, alternatively at least 2.25 times higher, alternatively at least 2.5 times higher, alternatively at least 2.75 times higher, alternatively at least 3 times higher; and/or (b) a CO₂ permeance that is at least 4 times higher than a CMS hollow fiber membrane resulting from pyrolysis of a single-layer precursor hollow fiber at the same temperature (when measured at 100 psia and 35° C.), alternatively at least 5 times higher, alternatively at least 7 times higher, alternatively at least 9 times higher, alternatively at least 10 times higher, alternatively at least 12 times higher.

Moreover, in some embodiments, the CMS hollow fiber membrane resulting from pyrolysis of a precursor fiber that has been treated by pre-pyro hybridization may have (a) a CO₂/CH₄ selectivity that is at least 1.5 times higher than the selectivity of a CMS hollow fiber membrane resulting from pyrolysis (at the same temperature) of the precursor fiber not treated by pre-pyro hybridization, alternatively at least 1.75 times higher, alternatively at least 2 times higher, alternatively at least 2.25 times higher, alternatively at least 2.5 times higher, alternatively at least 2.75 times higher, alternatively at least 3 times higher, alternatively at least 3.5 times higher, alternatively at least 4 times higher; and/or (b) a CO₂ permeance that is at least 50% of the CO₂ permeance of a CMS hollow fiber membrane resulting from pyrolysis (at the same temperature) of the precursor fiber not treated by pre-pyro hybridization, alternatively at least 55%, alternatively at least 60%, alternatively at least 65%, alternatively at least 70%, alternatively at least 75%. The precursor fiber may be a dual-layer precursor fiber prepared according to the present disclosure or the precursor fiber may be an asymmetric single-layer precursor fiber with or without a defective skin layer.

The ultrathin CMS hollow fibers disclosed in this invention are only evaluated for CO₂/CH₄ separations; however, it is clear that the ultrathin CMS hollow fiber platform can be extended to other molecular gas and liquid separations.

### Treatment of Thermally Re-Arranged Polymer Membranes

The pores and channels within a polymer film or fiber typically have a wide range of sizes, which render the polymer structures generally unsuitable for gas separation applications. In various embodiments, pyrolysis of a polymer material forms a carbon molecular sieve material having ordered pores. However, certain polymers may be treated to render the polymer itself, e.g. without pyrolysis, suitable for gas separation applications. Thermally re-arranged polymer membranes, also known as TR polymer membranes or TR polymer fibers, remedy the problem of variable pore sizes by thermally driving spatial rearrangement of rigid polymer chain segments in the glassy phase in order to produce pores having a more controlled size. These changes in the polymer structure are said to increase permeability and selectivity properties, rendering the polymer suitable for gas separation.

Preferred thermally re-arranged polymer membranes comprise aromatic polymers that are interconnected with heterocyclic rings. Examples include polybenzoxazoles, polybenzothiazoles, and polybenzimidazoles. Preferred thermally re-arranged polymer precursors comprise polyimides with ortho-positioned functional groups, such as for example HAB-6FDA, a polyimide having the following structure.

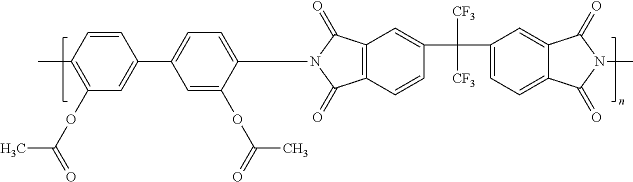

The phenylene-heterocyclic ring units in such materials have rigid chain elements and a high-torsional energy barrier to rotation between the two rings, which prevents indiscriminant rotation. Thermal re-arrangement of these polymers can thus be controlled to create pores having a narrow size distribution, rendering them useful for gas separation applications.

The temperature at which the thermal rearrangement occurs is generally lower than the temperatures used for pyrolysis, as pyrolysis would convert the polymer fiber into a carbon fiber. Polyimides, for example, are typically heated to a temperature between about 250° C. and about 500° C., more preferably between about 300° C. and about 450° . The heating of the polymers generally takes place in an inert atmosphere for a period of a number of hours Like polymers that are subjected to pyrolysis in order to form CMS hollow fiber membranes, the polymers that are subjected to thermal rearrangement may contain defects and/or packing irregularities, especially at the skin layer.

Accordingly, embodiments of the present disclosure are directed to the treatment of a polymer using the pre-pyro hybridization treatment described herein, which in the case of a thermally-rearranged polymer can be referred to as pre-arrangement hybridization (since these polymers are not subjected to pyrolysis). The pre-arrangement hybridization treatment may be performed in order to increase the gas separation selectivity of the resulting thermally rearranged polymeric membrane.

Treatment of the polymer material is performed in the same manner described above with respect to treatment of polymer precursor fibers that are then pyrolyzed to form asymmetric CMS hollow fiber membranes. The difference being that, after treatment, the treated polymer material is subjected to thermal re-arrangement, as is described above and known in the art, as opposed to pyrolysis. Embodiments of the present invention are also directed to thermally re-arranged polymer materials that have been subjected to pre-arrangement hybridization as described herein, and thereby have improved selectivities over the same thermally rearranged polymeric material that is not subjected to such a treatment.

In some embodiments, for example, the thermally rearranged polymeric membrane resulting from thermal rearrangement of a polymer that has been treated by pre-arrangement hybridization may have (a) a CO₂/CH₄ selectivity that is at least 1.5 times higher than the selectivity of a thermally rearranged polymeric membrane resulting from rearrangement (at the same temperature) of the polymer not treated by pre-arrangement hybridization, alternatively at least 1.75 times higher, alternatively at least 2 times higher, alternatively at least 2.25 times higher, alternatively at least 2.5 times higher, alternatively at least 2.75 times higher, alternatively at least 3 times higher, alternatively at least 3.5 times higher, alternatively at least 4 times higher; and/or (b) a CO₂ permeance that is at least 50% of the CO₂ permeance of a thermally rearranged polymeric membrane resulting from rearrangement (at the same temperature) of the polymer not treated by pre-arrangement hybridization, alternatively at least 55%, alternatively at least 60%, alternatively at least 65%, alternatively at least 70%, alternatively at least 75%.

### Various Aspects of the Present Disclosure

Aspects of the present disclosure relate to any combination of the following:

### Dual-Layer Precursor Fibers

1. A method for preparing a carbon molecular sieve hollow fiber membrane having a thin outer skin layer, the method comprising:

a. preparing a hollow polymer fiber having a core layer and a sheath layer; and

b. pyrolyzing the hollow polymer fiber to prepare a carbon molecular sieve hollow fiber membrane;

wherein the carbon molecular sieve hollow fiber membrane comprises a porous substrate layer and an outer skin layer, the outer skin layer having a thickness of 2 microns or less.

2. The method of aspect 1, wherein preparing the hollow polymer fiber comprises

i. coextruding a two-layer dope composition and a bore fluid through a spinneret into an air gap, and

ii. immersing the resulting fiber in an aqueous quench bath; wherein the two-layer dope composition comprises a core dope and a sheath dope.

3. The method of aspect 2, wherein the sheath dope has a thickness of 2 microns or less.

4. The method of aspect 1, wherein the core layer comprises one or more pore-forming chemicals.

5. The method of any one of aspects 2 to 3, wherein the core dope comprises one or more pore-forming chemicals, the one or more pore-forming chemicals being present at a concentration between 0.5 wt. % and 20 wt. % of the core dope.

6. The method of any one of aspects 4 and claim 5, wherein the one or more pore-forming chemicals comprises polyvinylpyrrolidone.

7. The method of any one of aspects 1 to 6, wherein the thickness of the outer skin layer is substantially the same as the thickness of the sheath layer.

8. The method of any one of aspects 1 to 7, wherein the core layer and the sheath layer comprise the same polymer or substantially the same polymer.

9. The method of aspect 8, wherein the polymer is a polyimide or a combination of polyimides.

10. The method of any one of aspect 8, wherein the carbon molecular sieve hollow fiber membrane comprises a CO₂ permeance, measured at 100 psia and 35° C., at least 4 times greater than the CO₂ permeance of a carbon molecular sieve hollow fiber membrane prepared from a single layer hollow polymer fiber under the same conditions.

11. The method of any one of aspects 1 to 7, wherein the core layer and the sheath layer comprise different polymers.

12. The method of any one of aspects 1 to 7, wherein the core layer comprises one or more polyimides and the sheath layer comprises the combination of one or more polyimides and one or more polyamides.

13. The method of any one of aspects 1 to 12, wherein the outer skin layer has a thickness of 1.5 microns or less.

14. The method of aspect 13, wherein the outer skin layer has a thickness of 1 micron or less.

15. The method of any one of aspects 1 to 14, further comprising treating the hollow polymer fiber prior to pyrolysis, the treatment comprising introducing an in-situ polymerized polymeric material into the pores of the sheath layer, thereby forming a hybrid sheath layer.

16. The method of aspect 15, wherein the in situ polymerized polymeric material comprises one or more polyamides, one or more polyimides, one or more polyamide-imides, or a combination thereof.

17. The method of any one of aspects 15 to 16, wherein the treatment comprises

a. contacting the hollow polymer fiber with a solution comprising a first monomer, and

b. contacting the hollow polymer fiber of step a. with a solution comprising a second monomer;

wherein the first monomer and the second monomer react to form an in-situ polymerized polymeric material.

18. The method of aspect 17, wherein the first monomer comprises a multi-functional amine and the second monomer comprises a multi-functional acyl halide.

19. The method of aspect 18, wherein the multi-functional amine comprises 2,5-diethyl-6-methyl-1,3-diamino benzene and the multi-functional acyl halide comprises trimesoyl chloride.

20. The method of aspect 15, wherein the in situ polymerized polymeric material comprises a polyamide or a combination of polyamides. Hybridization of outer layer

21. A method for preparing a carbon molecular sieve hollow fiber membrane having a dense skin layer, the method comprising:
a. providing a hollow polymer fiber;
b. treating the hollow polymer fiber to introduce an in situ polymerized polymeric material onto an outer surface of the hollow polymer fiber, into interstitial defects of the hollow polymer fiber, or both, thereby forming a hollow polymer fiber having a hybrid outer layer; and
c. pyrolyzing the treated hollow polymer fiber to prepare a carbon molecular sieve hollow fiber membrane.

22. The method of aspect 21, wherein the in situ polymerized polymeric material comprises one or more polyamides, one or more polyimides, one or more polyamide-imides, or a combination thereof.

23. The method of any one of aspects 21 and 22, wherein the hollow polymer fiber comprises one or more polyimides.

24. The method of any one of aspects 21 to 23, wherein treating the hollow polymer fiber comprises

i. contacting the hollow polymer fiber with a first solution comprising a first monomer;

ii. contacting the hollow polymer fiber of step i. with a second solution comprising a second monomer, wherein the first monomer and the second monomer react to form the in situ polymerized polymeric material.

25. The method of aspect 24, wherein the first monomer comprises a multi-functional amine and the second monomer comprises a multi-functional acyl halide.

26. The method of aspect 25, wherein the multi-functional amine comprises 2,5-diethyl-6-methyl-1,3-diamino benzene and the multi-functional acyl halide comprises trimesoyl chloride.

27. The method of any one of aspects 24 to 26, wherein the first solution comprises between 0.0001 wt. % and 1 wt. % of the first monomer and the second solution comprises between 0.0001 wt. % and 1 wt. % of the second monomer.

28. The method of aspect 27, wherein the first solution comprises between 0.001 wt. % and 0.1 wt. % of the first monomer and the second solution comprises between 0.001 wt. % and 0.1 wt. % of the second monomer.

29. The method of any one of aspects 24 to 28, wherein contacting the hollow polymer fiber with each of the first solution and the second solution comprises soaking the hollow polymer fiber in each solution.

30. The method of aspect 29, wherein the hollow polymer fiber is soaked in the first solution for 30 minutes or less and the hollow polymer fiber is soaked in the second solution for 30 minutes or less.

31. The method of any one of aspects 24 to 30, further comprising:
iii. drying the hollow polymer fiber of step ii. prior to pyrolysis. 32. The method of any one of aspects 21 to 30, wherein the hybrid outer layer has a thickness of 2 microns or less.

33. The method of aspect 32, wherein the hybrid outer layer has a thickness of 1 microns or less.

34. The method of any one of aspects 21 to 33, wherein the carbon molecular sieve hollow fiber membrane comprises a CO₂/CH₄ selectivity, measured at 100 psia and 35° C., that is at least double the CO₂/CH₄ selectivity of a carbon molecular sieve hollow fiber membrane prepared under the same conditions but without the treatment step.

35. The method of any one of aspects 21 to 34, wherein the hollow polymer fiber comprises a core layer and a sheath layer.

36. The method of aspect 35, wherein the sheath layer has a thickness of 2 microns or less.

37. The method of any one of aspects 35 and 36, wherein providing the hollow polymer fiber comprises:

i. coextruding a two-layer dope composition and a bore fluid through a spinneret into an air gap, and

ii. immersing the resulting fiber in an aqueous quench bath;

wherein the two-layer dope composition comprises a core dope and a sheath dope.

38. The method of aspect 37, wherein the core dope comprises one or more pore-forming chemicals.

39. The method of aspect 38, wherein the one or more pore-forming chemicals comprises polyvinylpyrrolidone.

40. The method of any one of aspects 21 to 34, wherein the hollow polymer fiber is an asymmetric single-layer fiber with a skin layer.

41. The method of aspect 40, wherein providing the hollow polymer fiber comprises:

i. coextruding a spinning dope and a bore fluid through a spinneret into an air gap, and

ii. immersing the resulting fiber in an aqueous quench bath;

42. The method of aspect 41, wherein the spinning dope comprises one or more pore-forming chemicals.

43. The method of any one of aspects 21 to 34, wherein the hollow polymer fiber is a single-layer fiber with porous surface and without a skin layer.

44. The method of aspect 43, wherein providing the hollow polymer fiber comprises:

i. coextruding a spinning dope and a bore fluid through a spinneret, and

ii. immersing the resulting fiber in an aqueous quench bath; 45. The method of aspect 44, wherein the spinning dope comprises one or more pore-forming chemicals.

### Combination

46. A method for preparing a carbon molecular sieve hollow fiber membrane having improved gas separation properties, the method comprising:
a. preparing a hollow polymer fiber having a core layer and a sheath layer;
b. treating the hollow polymer fiber to introduce an in-situ polymerized polymeric material onto an outer surface of the hollow polymer fiber, into interstitial defects in the sheath layer, or both; and
c. pyrolyzing the hollow polymer fiber to prepare a carbon molecular sieve hollow fiber membrane;
wherein the carbon molecular sieve hollow fiber membrane comprises a porous substrate layer and a dense outer skin layer, the dense outer skin layer having a thickness of 2 microns or less.

47. The method of aspect 46, wherein preparing the hollow polymer fiber comprises:

i. coextruding a two-layer dope composition and a bore fluid through a spinneret into an air gap, and

ii. immersing the resulting fiber in an aqueous quench bath;

wherein the two-layer dope composition comprises a core dope and a sheath dope.

48. The method of aspect 47, wherein the core dope comprises one or more pore-forming chemicals.

49. The method of aspect 48, wherein the one or more pore-forming chemicals comprises polyvinylpyrrolidone.

50. The method any one of aspects 46 to 49, wherein the cross-linked polymeric material comprises one or more polyamides, one or more polyimides, one or more polyamide-imides, or a combination thereof.

51. The method of any one of aspects 46 to 50, wherein the treatment comprises

a. contacting the hollow polymer fiber with a solution comprising a first monomer, and

b. contacting the hollow polymer fiber of step a. with a solution comprising a second monomer;

wherein the first monomer and the second monomer react to form the in-situ polymerized polymeric material.

52. The method of aspect 51, wherein the first monomer comprises a multi-functional amine and the second monomer comprises a multi-functional acyl halide.

53. The method of aspect 52, wherein the multi-functional amine comprises 2,5-diethyl-6-methyl-1,3-diamino benzene and the multi-functional acyl halide comprises trimesoyl chloride.

54. The method of any one of aspects 51 to 53, wherein the each of the first monomer and the second monomer are present in solution at a concentration between 0.001 wt. % and 0.1 wt. %.

55. The method of any one of aspects 51 to 54, wherein contacting the hollow polymer fiber with each of the solutions comprises soaking the hollow polymer fiber in each solution.

56. The method of any one of aspects 46 to 55, wherein the core layer and the sheath layer comprise a polyimide or a combination of polyimides.

57. The method of any one of aspects 46 to 56, wherein the core layer and the sheath layer comprise the same polymer or substantially the same polymer.

58. The method of any one of aspects 46 to 57, wherein the outer skin layer has a thickness of 1.5 microns or less.

59. The method of aspect 58, wherein the outer skin layer has a thickness of 1 micron or less.

60. The method of any one of aspects 46 to 59, wherein when pyrolysis is performed at about 550° C., the carbon molecular sieve hollow fiber membrane comprises a CO₂/CH₄ selectivity of at least 15 and a CO₂ permeance of at least 800, measured at 100 psia and 35° C.

61. The method of any one of aspects 46 to 59, wherein when pyrolysis is performed at about 650° C., the carbon molecular sieve hollow fiber membrane comprises a CO₂/CH₄ selectivity of at least 65 and a CO₂ permeance of at least 150, measured at 100 psia and 35° C.

62. The method of any one of aspects 46 to 59, further comprising controlling the thickness of the sheath layer, the amount of in-situ polymerized polymeric material introduced, or both, to provide a carbon molecular sieve hollow fiber membrane having a desired combination of gas permeance and selectivity properties.

### Additional Aspects

63. The carbon molecular sieve hollow fiber membrane prepared by any one of aspects 1 to 62.

64. A process for separating at least a first gas component and a second gas component comprising:
a. providing a carbon molecular sieve membrane prepared by any one of aspects 1 to 62; and
b. contacting a gas stream comprising at least a first gas component and a second gas component with the carbon molecular sieve membrane to produce
i. a retentate stream having a reduced concentration of the first gas component, and
ii. a permeate stream having an increased concentration of the first gas component;

65. The process of aspect 64, wherein the first gas component is CO₂, H₂S, or a mixture thereof and the second gas component is CH₄.

66. The process of aspect 64, wherein the first gas component is ethylene or propylene and the second gas component is ethane or propane.

67. The process of aspect 64, wherein the first gas component is oxygen and the second gas component is nitrogen.

68. The process of aspect 64, wherein the first gas component is carbon dioxide and the second gas component is nitrogen.

69. The process of aspect 64, wherein the first gas component is n-butane and the second gas component is iso-butane.

70. The process of aspect 64, wherein the first gas component is iso-butylene and the second gas component is iso-butane.

71. The process of aspect 64, wherein the first gas component is butadiene and the second gas component is n-butane, 1-butylene, 2-butylene, iso-butane, iso-butylene, or a mixture thereof.

72. The process of aspect 64, wherein the first gas component is n-pentane and the second gas component is iso-pentane, neo-pentane, or a mixture thereof.

73. The process of aspect 64, wherein the first gas component is n-hexane and the second gas component is 2-methylpentane, 3-methylpentane, 2,3-dimethylbutane, 2,2-dimethylbutane, or a mixture thereof.

74. The process of aspect 64, wherein the first gas component is para-xylene and the second gas component is ortho-xylene, meta-xylene, ethylbenzene, or a mixture thereof.

75. The process of aspect 64, wherein the first gas component is hydrogen and the second gas component is methane, ethane, propane, n-butane, isobutane, or a mixture thereof.

76. The process of aspect 64, wherein the first gas component is helium and the second gas component is methane.

77. The process of aspect 64, wherein the first gas component is hydrogen and the second gas component is ethylene, propylene, 1-butylene, 2-butylene, iso-butylene, butadiene, or a mixture thereof.

78. A process for separating acid gas components from a natural gas stream comprising:

a. providing a carbon molecular sieve membrane prepared by any one of aspects 1 to 62; and

b. contacting a natural gas stream having one or more acid gas components with the carbon molecular sieve membrane to produce

i. a retentate stream having a reduced concentration of acid gas components, and

ii. a permeate stream having an increased concentration of acid gas components.

79. A carbon molecular sieve hollow fiber membrane comprising a porous substrate layer and an outer skin layer, wherein the outer skin layer has a thickness less than 2 microns.

80. The carbon molecular sieve hollow fiber membrane of aspect 79, wherein the outer skin layer has a thickness less than 1.5 microns.

81. The carbon molecular sieve hollow fiber membrane of aspect 80, wherein the outer skin layer has a thickness less than 1 micron.

82. The carbon molecular sieve hollow fiber membrane of aspect 81, wherein the outer skin layer has a thickness less than 0.5 micron.

83. The carbon molecular sieve hollow fiber membrane of any one of aspects 79 to 82, further comprising a sharp interface between the porous substrate layer and the outer skin layer.

### Pre-Rearrangement Hybridization of Thermally-Rearranged Polymers

84. A method for preparing a thermally rearranged polymeric membrane having, the method comprising:
a. providing a hollow polymer fiber;
b. treating the hollow polymer fiber to introduce an in situ polymerized polymeric material onto an outer surface of the hollow polymer fiber, into interstitial defects of the hollow polymer fiber, or both, thereby forming a hollow polymer fiber having a hybrid outer layer; and
c. thermally rearranging the treated hollow polymer fiber to prepare a thermally rearranged polymeric membrane.

85. The method of aspect 84, wherein the in situ polymerized polymeric material comprises one or more polyamides, one or more polyimides, one or more polyamide-imides, or a combination thereof.

86. The method of any one of aspects 84 and 85, wherein the hollow polymer fiber comprises one or more polyimides.

87. The method of any one of aspects 84 to 86, wherein treating the hollow polymer fiber comprises

i. contacting the hollow polymer fiber with a first solution comprising a first monomer;

ii. contacting the hollow polymer fiber of step i. with a second solution comprising a second monomer, wherein the first monomer and the second monomer react to form the in situ polymerized polymeric material.

88. The method of aspect 87, wherein the first monomer comprises a multi-functional amine and the second monomer comprises a multi-functional acyl halide.

89. The method of aspect 88, wherein the multi-functional amine comprises 2,5-diethyl-6-methyl-1,3-diamino benzene and the multi-functional acyl halide comprises trimesoyl chloride.

90. The method of any one of aspects 87 to 89, wherein the first solution comprises between 0.0001 wt. % and 1 wt. % of the first monomer and the second solution comprises between 0.0001 wt. % and 1 wt. % of the second monomer.

91. The method of aspect 90, wherein the first solution comprises between 0.001 wt. % and 0.1 wt. % of the first monomer and the second solution comprises between 0.001 wt. % and 0.1 wt. % of the second monomer.

92. The method of any one of aspects 87 to 91, wherein contacting the hollow polymer fiber with each of the first solution and the second solution comprises soaking the hollow polymer fiber in each solution.

93. The method of aspect 92, wherein the hollow polymer fiber is soaked in the first solution for 30 minutes or less and the hollow polymer fiber is soaked in the second solution for 30 minutes or less.

94. The method of any one of aspects 87 to 93, further comprising:
iii. drying the hollow polymer fiber of step ii. prior to thermal rearrangement.

95. The method of any one of aspects 84 to 94, wherein the hybrid outer layer has a thickness of 2 microns or less.

96. The method of aspect 95, wherein the hybrid outer layer has a thickness of 1 microns or less.

97. The method of any one of aspects 84 to 96, wherein the thermally rearranged polymeric membrane comprises a CO₂/CH₄ selectivity, measured at 100 psia and 35° C., that is at least double the CO₂/CH₄ selectivity of a thermally rearranged polymeric membrane prepared under the same conditions but without the treatment step.

98. The thermally-rearranged polymeric membrane produced by any of claims 84 to 97.

99. A process for separating at least a first gas component and a second gas component comprising:

a. providing a thermally rearranged polymeric membrane prepared by any one of aspects 84 to 97; and

b. contacting a gas stream comprising at least a first gas component and a second gas component with the thermally rearranged polymeric membrane to produce

i. a retentate stream having a reduced concentration of the first gas component, and

ii. a permeate stream having an increased concentration of the first gas component; 100. The process of aspect 99, wherein the first gas component and the second gas component are selected from those listed in aspects 65 to 78.

It can be seen that the described embodiments provide unique and novel methods for preparing CMS hollow fiber membranes for gas separation applications that have a number of advantages over those in the art. While there is shown and described herein certain specific structures embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. A method for preparing a carbon molecular sieve hollow fiber membrane having a thin outer skin layer, the method comprising:
a. preparing a hollow polymer fiber having a core layer and a sheath layer; and
b. pyrolyzing the hollow polymer fiber to prepare a carbon molecular sieve hollow fiber membrane;
wherein the carbon molecular sieve hollow fiber membrane comprises a porous substrate layer and an outer skin layer, the outer skin layer having a thickness of 2 microns or less.

2. The method of claim 1, wherein preparing the hollow polymer fiber comprises
i. coextruding a two-layer dope composition and a bore fluid through a spinneret into an air gap, and
ii. immersing the resulting fiber in an aqueous quench bath;
wherein the two-layer dope composition comprises a core dope and a sheath dope.

3. The method of claim 2, wherein the sheath dope has a thickness of 2 microns or less.

4. The method of claim 1, wherein the core layer comprises one or more pore-forming chemicals.

5. The method of claim 2, wherein the core dope comprises one or more pore-forming chemicals, the one or more pore-forming chemicals being present at a concentration between 0.5 wt. % and 20 wt. % of the core dope.

6. The method of claim 4, wherein the one or more pore-forming chemicals comprises polyvinylpyrrolidone.

7. The method of claim 1, wherein the thickness of the outer skin layer is substantially the same as the thickness of the sheath layer.

8. The method of claim 1, wherein the core layer and the sheath layer comprise the same polymer or substantially the same polymer.

9. The method of claim 8, wherein the polymer is a polyimide or a combination of polyimides.

10. The method of claim 8, wherein the carbon molecular sieve hollow fiber membrane comprises a CO₂ permeance, measured at 100 psia and 35° C., at least 4 times greater than the CO₂ permeance of a carbon molecular sieve hollow fiber membrane prepared from a single layer hollow polymer fiber under the same conditions.

11. The method of claim 1, wherein the core layer and the sheath layer comprise different polymers.

12. The method of claim 1, wherein the core layer comprises one or more polyimides and the sheath layer comprises the combination of one or more polyimides and one or more polyamides.

13. The method of claim 1, wherein the outer skin layer has a thickness of 1.5 microns or less.

14. The method of claim 13, wherein the outer skin layer has a thickness of 1 micron or less.

15. The method of claim 1, further comprising treating the hollow polymer fiber prior to pyrolysis, the treatment comprising introducing an in-situ polymerized polymeric material into the pores of the sheath layer, thereby forming a hybrid sheath layer.

16. The method of claim 15, wherein the in situ polymerized polymeric material comprises one or more polyamides, one or more polyimides, one or more polyamide-imides, or a combination thereof.

17. The method of claim 15, wherein the treatment comprises
a. contacting the hollow polymer fiber with a solution comprising a first monomer, and
b. contacting the hollow polymer fiber of step a. with a solution comprising a second monomer;
wherein the first monomer and the second monomer react to form an in-situ polymerized polymeric material.

18. The method of claim 17, wherein the first monomer comprises a multi-functional amine and the second monomer comprises a multi-functional acyl halide.

19. The method of claim 18, wherein the multi-functional amine comprises 2,5-diethyl-6-methyl-1,3-diamino benzene and the multi-functional acyl halide comprises trimesoyl chloride.

20. The carbon molecular sieve hollow fiber membrane prepared by claim 1.

21. A process for separating at least a first gas component and a second gas component comprising:
a. providing a carbon molecular sieve membrane prepared by claim 1; and
b. contacting a gas stream comprising at least a first gas component and a second gas component with the carbon molecular sieve membrane to produce
i. a retentate stream having a reduced concentration of the first gas component, and
ii. a permeate stream having an increased concentration of the first gas component;

22. The process of claim 21, wherein the first gas component is CO₂, H₂S, or a mixture thereof and the second gas component is CH₄.

23. The process of claim 21, wherein the first gas component is ethylene or propylene and the second gas component is ethane or propane.

24. The process of claim 21, wherein the first gas component is oxygen and the second gas component is nitrogen.

25. The process of claim 21, wherein the first gas component is carbon dioxide and the second gas component is nitrogen.

26. A process for separating acid gas components from a natural gas stream comprising:
a. providing a carbon molecular sieve membrane prepared by claim 1; and
b. contacting a natural gas stream having one or more acid gas components with the carbon molecular sieve membrane to produce
i. a retentate stream having a reduced concentration of acid gas components, and
ii. a permeate stream having an increased concentration of acid gas components.
